(19) United States
(12) Patent Application Publication
(10) Pub. No.: US 2010/0262008 A1
(43) Pub. Date: Oct. 14, 2010

(54) ROBOTIC ULTRASOUND SYSTEM WITH MICROADJUSTMENT AND POSITIONING CONTROL USING FEEDBACK RESPONSIVE TO ACQUIRED IMAGE DATA

(75) Inventor: David N. Roundhill, Woodinville, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/747,238

(22) PCT Filed: Dec. 8, 2008
(86) PCT No.: PCT/IB08/55151

§ 371 Date: Jun. 10, 2010

Related U.S. Application Data
(60) Provisional application No. 61/013,330, filed on Dec. 13, 2007.

(51) Int. Cl. A61B 8/00

(52) U.S. Cl. 600/453

(57) ABSTRACT

An imaging system includes a diagnostic ultrasound front end module, the front end module including a transducer, a robotic armature (2), and a controller (4) electrically coupled to each of the front end module and the robotic armature. The controller is configured to employ the robotic armature to move the transducer relative to an anatomical structure, including wherein the controller is operable in a feedback control mode to detect key attributes in an acquired image or data set received from the front end module, calculate a desired adjustment to the position of the transducer based on the key attributes detection, and employ the robotic armature to apply the desired position adjustment.

The present disclosure is directed to medical diagnostic imaging systems and methods and, more particularly, to systems and methods for moving and controlling the motion of a transducer during ultrasound examinations.

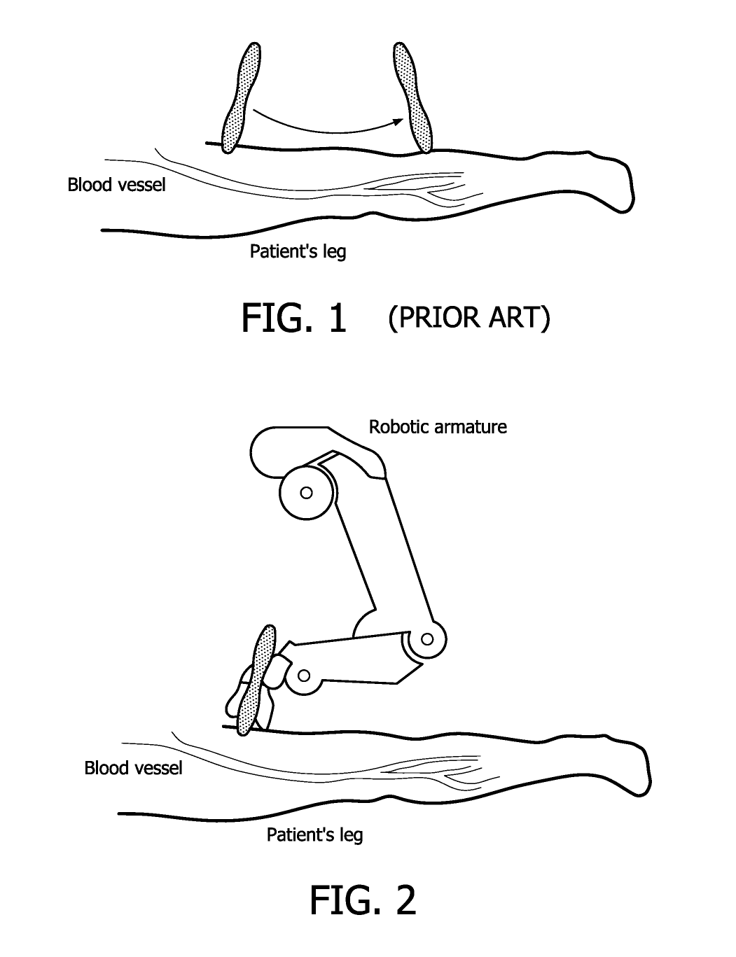

One of the attributes of a good sonographer is the ability to “micromanipulate” the position and spatial orientation of the ultrasound transducer to ensure an optimal signal, be it for gray scale imaging, color flow, spectral Doppler, or any traditional or modern imaging application. Some ultrasound imaging applications, however, can present particular challenges. For example, and as illustrated in FIG. 1, acquiring anatomic and flow data from the peripheral vasculature of a limb by means of an externally-manipulated transducer can be quite laborious. Various tasks involved with such a procedure, such as, for example, spatially orienting and reorienting the transducer as necessary with respect to the limb and the particular bodily structure under, applying an appropriate level of force when pressing the transducer against the skin and underlying tissue of the limb, and translating the transducer along the length of the limb along a unique path defined by the particular bodily structure under examination, are commonly performed manually by the use of a hand-held transducer head, putting the skills and talents of even the very best technicians to the test.

Despite efforts to date, a need remains for ultrasound data collection and manipulation solutions that are effective to enhance the quality and/or efficiency of ultrasound examinations, and/or to assist sonographers in conducting such examinations. These and other needs are satisfied by the disclosed systems and methods, as will be apparent from the description which follows.

In accordance with exemplary embodiments of the present disclosure, an imaging system is disclosed. The imaging system includes a diagnostic ultrasound front end module, the front end module including a transducer, a robotic armature, and a controller electrically coupled to each of the front end module and the robotic armature. The controller is configured to employ the robotic armature to move the transducer relative to an anatomical structure, including wherein the controller is operable in a feedback control mode to detect key attributes in an acquired image or data set received from the front end module, calculate a desired adjustment to the position of the transducer based on the key attributes detection, and employ the robotic armature to apply the desired position adjustment. The system may also include a user control electrically coupled to the controller, the user control being configured to permit a user to operate the robotic armature using haptic feedback. The controller may incorporate a feedback control mechanism that applies large translations of the transducer to follow anatomy detected via image analysis, applies small translations of the transducer in direct response to the detected key attributes, and/or applies small translations of the transducer via small perturbations away from a predefined position. The controller may further incorporate beamforming control, coarse and fine control of a robotic armature using haptic feedback., and/or applied force sensing and a feedback to modulate a force applied by the robotic armature to the patient via the transducer. The robotic armature may include an integrated force sensor electrically coupled to the controller and used to orient and place the transducer on or within the patient. The system may further include a diagnostic imaging system back end module electrically coupled to the controller and including a user interface, and/or a scanning control interface processor electrically coupled to the front end module, the controller, and the back end module.

In accordance with exemplary embodiments of the present disclosure, a method for adjusting the position of a transducer with respect to an anatomical structure is disclosed. The method includes using the transducer to acquire an image or a data set corresponding to the anatomical structure, detecting key attributes in the acquired image or data set, calculating a desired adjustment to the position of the transducer based on the key attributes detection, and repositioning the transducer in accordance with the desired adjustment. Repositioning the transducer in accordance with the desired adjustment may include employing a robotic armature to so reposition the transducer, applying large translations of the transducer to follow anatomy detected via image analysis, applying small translations of the transducer in direct response to the detected key attributes, and/or applying small translations of the transducer via small perturbations away from a predefined position.

Additional features, functions and benefits of the disclosed systems and methods will be apparent from the description which follows, particularly when read in conjunction with the appended figures.

To assist those of skill in the art in making and using the disclosed systems and methods for rendering an ultrasound volume, reference is made to the accompanying figures, wherein:

FIG. 1 illustrates a prior art arrangement for using an externally-manipulated transducer to acquiring anatomic and flow data from the peripheral vasculature of a limb;

FIG. 2 illustrates an image acquisition system in accordance with embodiments of the present disclosure; and

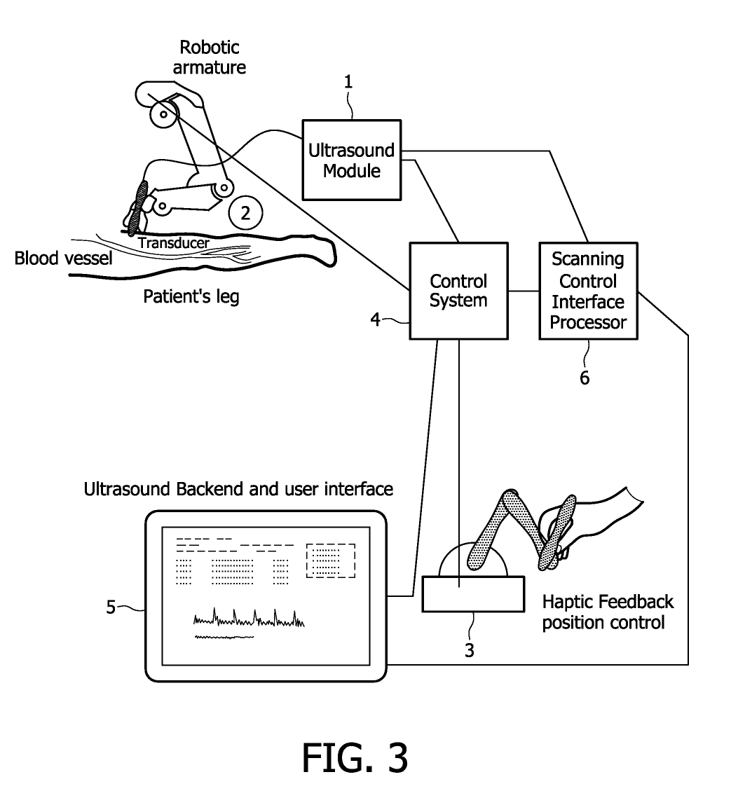

FIG. 3 illustrates an ultrasound system in accordance with embodiments of the present disclosure.

In accordance with exemplary embodiments of the present disclosure, an arrangement of components constituting an enhanced ultrasonic imaging system is provided. Such an arrangement takes advantage of the flexibility of translation and the precision of movement offered by a robotic armature to enhance the repeatability, reliability, and speed of ultrasound examinations, and to reduce the level of skill and/or manual dexterity required of sonographers conducting such examinations. Other benefits may include providing the ability to conduct ultrasound examinations remotely.

The present disclosure sets forth technology cooperative with that set forth within two additional Philips-owned invention disclosures. One such disclosure was incorporated in nonprovisional U.S. patent application Ser. No. 10/536,642 entitled “Segmentation Tool For Identifying Flow Regions In An Image System”, which application was published by the USPTO on May 11, 2006 as U.S. Patent Application Publication No. US 2006/0098853. (A full copy of this publication is included as part of the present disclosure (see Appendix I below).) In U.S. Patent Application Publication No. US 2006/0098853, the inventors describe, inter alia, a means of first identifying a region where flow is present and then automatically identifying a region in which to target spectral Doppler data acquisition by appropriate steering of the acoustic beamforming within the field of view of a 2 or 3D region. With respect to the other such disclosure, which is not yet filed as a patent application but is tentatively entitled “Haptic Feedback Control Of Robotic Armature for Ultrasound Scanning”, the inventor describes a means of remotely controlling a robotic arm to manipulate the placement of a transducer in response to applied force using a haptic control interface.

As indicated above, a good sonographer is capable of “micromanipulating” the position and orientation of the ultrasound transducer to ensure an optimal signal for gray scale, color flow or spectral Doppler, among other imaging applications. In accordance with the present disclosure, this ability may be automated or semi-automated in at least some instances via the use of a robotic arm for translating, orienting, reorienting and/or otherwise manipulating the transducer, including wherein the robotic arm accomplishes such transducer manipulation in response to one or both of human operator commands and computer-based algorithmic control.

Turning now to FIG. 2, an image acquisition system is set forth in accordance with embodiments of the present disclosure including a transducer, and a robotic arm and control feedback mechanism used to keep the transducer in contact with a patient's limb and centrally placed on a vessel lumen, and to translate the transducer along the length of the limb to an extent necessary to capture the desired image data. In accordance with at least some embodiments of the present disclosure, the translation of the transducer along the limb may be in response to a continuous input from the sonographer/technician. In accordance with at least some other embodiments, the translation of the transducer along the limb may be more fully automated, whereby the sonographer/technician initiates the scan and then monitors its progress.

The control system may incorporate edge detection of the blood vessel lumen and apply appropriate positional corrections to ensure that the transducer remains centrally positioned. In exams of this kind, it is common practice to acquire spectral Doppler data at key locations such as around points where the vessel bifurcates, or in the location of an athlosclerotic plaque. Such locations can be automatically detected both by computer aided analysis of the gray scale anatomic data as well as the detection of turbulence and velocity parameters present in the color flow data. Automatic placement of a Doppler sample volume and automatic collection around that position may then be facilitated by a combination of micro positioning the transducer using the robotic arm and adjustment of the beamforming (see U.S. Patent Application Publication No. US 2006/0098853, a copy of which is set forth herein as Appendix I). In accordance with embodiments of the present disclosure, such a capability is further enabled via the transducer and ultrasound system is equipped to acquire three-dimensional (3D) image data.

Referring to FIG. 3, an ultrasound system is illustrated in accordance with embodiments of the present disclosure. The system may include one or more, or all, of the following components: 1.) a diagnostic ultrasound system “front end”, including transducer; 2.) a robotic armature with integrated force sensors used to orient and place the imaging transducer on or within the patient; 3.) a user control for the robotic armature that uses haptic feedback; 4.) a control system that detects key attributes in an acquired image (or data set) and: a.) incorporates a feedback control mechanism that applies large translations of the transducer to follow anatomy detected via image analysis, b.) incorporates a feedback control system that applies small translations of the transducer either in direct response to the detected attributes or via small perturbations away from a user defined position, c.) incorporates beamforming control as disclosed in U.S. Patent Application Publication No. U.S. 20060098853, d.) incorporates coarse and fine control of a robotic armature using haptic feedback, and/or e.) incorporates applied force sensing and a feedback to modulate the force applied to the patient via the transducer; 5.) a diagnostic ultrasound system “back end”; and/or 6.) a scanning control interface processor.

The systems and methods of the present disclosure are particularly useful for acquiring, processing, and/or using as feedback for transducer motion control, ultrasound image data. However, the disclosed systems and methods are susceptible to many variations and alternative applications, without departing from the spirit or scope of the present disclosure.

1. An imaging system, comprising:
a diagnostic ultrasound front end module, the front end module including an ultrasound transducer;
a robotic armature; and
a controller electrically coupled to each of the front end module and the robotic armature, the controller being configured to employ the robotic armature to move the ultrasound transducer relative to an anatomical structure, including wherein the controller is operable in a feedback control mode to detect key attributes in an acquired image and data set received from the front end module, calculate a desired adjustment to the a position of the transducer based on the key attributes detection, and employ the robotic armature to apply the desired position adjustment, and facilitate an automatic placement of a Doppler sample volume and automatic collection around that placement by a combination of micro positioning the transducer using the robotic armature and an adjustment of beamforming.

2. An imaging system in accordance with claim 1, further comprising a user control electrically coupled to the controller, the user control being configured to permit a user to operate the robotic armature using haptic feedback.

3. An imaging system in accordance with claim 1, wherein the controller incorporates a feedback control mechanism that applies large translations of the transducer to follow anatomy detected via image analysis.

4. An imaging system in accordance with claim 1, wherein the controller incorporates a feedback control mechanism that applies small translations of the transducer in direct response to the detected key attributes.

5. An imaging system in accordance with claim 1, wherein the controller incorporates a feedback control mechanism that applies small translations of the transducer via small perturbations away from a predefined position.

6. An imaging system in accordance with claim 1, wherein the controller incorporates beamforming control.

7. An imaging system in accordance with claim 1, wherein the controller incorporates coarse and fine control of the robotic armature using haptic feedback.

8. An imaging system in accordance with claim 1, wherein the controller incorporates applied force sensing and a feedback to modulate a force applied by the robotic armature to the patient via the transducer.

9. An imaging system in accordance with claim 1, wherein the robotic armature includes an integrated force sensor electrically coupled to the controller and used to orient and place the transducer on or within the patient.

10. An imaging system in accordance with claim 1, further including a diagnostic imaging system back end module electrically coupled to the controller and including a user interface.

11. An imaging system in accordance with claim 10, further including a scanning control interface processor electrically coupled to the front end module, the controller, and the back end module.

12. An imaging system in accordance with claim 1, further including a scanning control interface processor electrically coupled to the front end module and the controller.

13. A method for adjusting the position of an ultrasound transducer with respect to an anatomical structure, the method comprising:
using the transducer coupled to a robotic armature to acquire an image and a data set corresponding to the anatomical structure;
detecting key attributes in the acquired image and data set;
calculating a desired adjustment to the position of the transducer based on the key attributes detection;
repositioning the transducer via the robotic armature in accordance with the desired adjustment; and
facilitating an automatic placement of a Doppler sample volume and automatic collection around that placement by a combination of micro positioning the transducer using the robotic armature and an adjustment of beamforming.

14. (canceled)

15. A method for adjusting the position of a transducer in accordance with claim 13, wherein repositioning the transducer in accordance with the desired adjustment includes applying large translations of the transducer to follow anatomy detected via image analysis.

16. A method for adjusting the position of a transducer in accordance with claim 13, wherein repositioning the transducer in accordance with the desired adjustment includes applying small translations of the transducer in direct response to the detected key attributes.

17. A method for adjusting the position of a transducer in accordance with claim 13, wherein repositioning the transducer in accordance with the desired adjustment includes applying small translations of the transducer via small perturbations away from a predefined position.
